(19) United States
(12) Patent Application Publication
(10) Pub. No.: US 2018/0199822 A1
(43) Pub. Date: Jul. 19, 2018

(54) BLOOD PRESSURE MEASUREMENT METHOD AND BLOOD PRESSURE MEASUREMENT DEVICE APPLYING THE SAME

(71) Applicant: Cheng Uei Precision Industry Co., Ltd., New Taipei City (TW)

(72) Inventor: James Lee, New Taipei City (TW)

(21) Appl. No.: 15/408,444

(22) Filed: Jan. 18, 2017

(51) Int. Cl. A61B 5/021; A61B 5/00; A61B 5/0402

(52) U.S. Cl. CPC A61B 5/021; A61B 5/7225; A61B 5/7275; A61B 5/02108; A61B 5/0402

(57) ABSTRACT

A blood pressure measurement method includes specific steps. Sample and store photoplethysmography signals and electrocardiosignals. Measure blood pressure to get a plurality of values of systolic blood pressure and diastolic blood pressure. Synchronize the electrocardiosignals and the photoplethysmography signals, and calculate a time interval between an electrocardiosignal feature point and a photoplethysmography signal feature point. Establish a formula for calculating the value of the systolic blood pressure: SBP=a1×PWV+b1×BMI+c1. Establish a formula for calculating the value of the diastolic blood pressure: DBP=d1×SBP+e1. Put the time interval between the electrocardiosignal feature point and the photoplethysmography signal feature point into the formula for calculating the value of the systolic blood pressure. Put the value of the systolic blood pressure into the formula for calculating the value of the diastolic blood pressure.

### BACKGROUND OF THE INVENTION

### 1. Field of the Invention

The present invention generally relates to a human physiological information measurement method, and more particularly to a blood pressure measurement method and a blood pressure measurement device applying the same.

### 2. The Related Art

A cardiovascular disease is a major health threatening faced by current mankind. Blood pressures can reflect function situations of a heart and blood vessels of a human body, and the blood pressures are important basises of diagnosing diseases clinically, observing therapeutic effects and prognosis judgements. Flowing blood generates lateral pressure on a unit blood vessel wall is called the blood pressure. The blood pressure is a combined action result of ejecting blood by a heart chamber and a peripheral resistance.

The blood pressure includes an arterial pressure and a venous pressure. Usually, the blood pressure refers to the arterial pressure. The arterial pressure is closely associated with a heart function and a peripheral vessel condition. The blood pressure has a continuous change in every cardiac cycle. When the heart chamber is contracted, the blood flows into an artery vessel from the heart chamber, a pressure on the artery vessel exerted by the blood is highest, at the moment, the pressure on the artery vessel is called a systolic pressure; when the heart chamber is diastolic, the artery vessel is elastically contracted, the blood still continues flowing frontward slowly, but the blood pressure is lowered, at the moment, the pressure on the artery vessel is called a diastolic pressure.

Because blood pressure parameters are affected by a physical condition, a environment condition, a biorhythm and other many factors, results measured singly or results measured intermittently have larger differences. A continuous measurement method is capable of being applied in every cardiac cycle to measure the blood pressure, and the continuous measurement method has a more important significance in a clinic research and other medical researches.

A current continuous blood pressure measurement method includes an invasive continuous blood pressure measurement method and a noninvasive continuous blood pressure measurement method. An intra-arterial catheter method is the invasive continuous blood pressure measurement method. The invasive continuous blood pressure measurement method is a golden standard in the blood pressure measurement. But it need prepare for a longer time to use the invasive continuous blood pressure measurement method to measure the blood pressure, and a health complication is easily caused, so the invasive continuous blood pressure measurement method is seldom applied, except for being applied in measuring the blood pressures of severe patients and the blood pressure measurement before a major operation.

The noninvasive blood pressure measurement method is a common measurement method in a clinic application and basic medicine. The noninvasive blood pressure measurement method is mostly an auscultatory method, an oscillographic method, an arterial tonometry, a volume-compensation method or other measurement method. Most of the noninvasive blood pressure measurement methods all need inflatable cuffs. A discomfort feeling and inflation time generated by using the inflatable cuff will bring difficulties to the continuous blood pressure measurement. Stimulus brought to subjects by the inflatable cuffs and inflation pressure will also affect measurement results.

However, most of the noninvasive measurement methods all use the inflatable cuffs, so the continuous blood pressure measurement has no way of being realized, and the stimulus brought to the subjects by the inflatable cuffs and the inflation pressure will also affect the measurement results.

Thus, it is essential to provide an innovative blood pressure measurement method, and an innovative blood pressure measurement device applying the innovative blood pressure measurement method to measure blood pressure. The innovative blood pressure measurement device has no need of using an inflatable cuff, and is capable of being worn for a long time. The innovative blood pressure measurement device is capable of noninvasively measuring and recording blood pressure values of subjects continuously.

### SUMMARY OF THE INVENTION

An object of the present invention is to provide a blood pressure measurement method. Specific steps of the blood pressure measurement method are described as follows. Sample and store photoplethysmography signals and electrocardiosignals from plural different subjects. Measure blood pressure of the plural different subjects to get a plurality of values of systolic blood pressure and diastolic blood pressure. Synchronize the electrocardiosignals and the photoplethysmography signals of each of the different subjects, and calculate a time interval between an electrocardiosignal feature point and a photoplethysmography signal feature point of each of the different subjects. Establish a formula for calculating the value of the systolic blood pressure of each of the subjects: SBP=a1×PWV+b1×BMI+c1. SBP is the value of the systolic blood pressure of each of the subjects; PWV is a pulse wave velocity, PWV=Height/(2×PTT), PTT is a pulse transit time, namely the time interval between the electrocardiosignal feature point and the photoplethysmography signal feature point of each of the subjects, Height is a height of each of the subjects; BMI is a body mass index of each of the subjects; a1 and b1 are two different coefficients, c1 is a constant. Calculate a1, b1 and c1 by means of obtained values of the systolic blood pressure, PTT, Height and BMI of the different subjects applying a predicted model of monadic linear regression analysis method. Establish a formula for calculating the value of the diastolic blood pressure of each of the subjects: DBP=d1×SBP+e1, DBP is the value of the diastolic blood pressure of each of the subjects, SBP is the value of the systolic blood pressure of each of the subjects, d1 and e1 are respectively two different coefficients. Calculate d1 and e1 by means of the obtained values of the systolic blood pressure and obtained values of the diastolic blood pressure of the different subjects applying the predicted model of monadic linear regression analysis method. Sample and store photoplethysmography signals and electrocardiosignals of a current user, synchronize the electrocardiosignals and the photoplethysmography signals of the current user, calculate a time interval between an electrocardiosignal feature point and a photoplethysmography signal feature point of the current user. Values of Height and BMI of the current user, and put the time interval between the electrocardiosignal feature point and the photoplethysmography signal feature point of a current user into the formula for calculating the value of the systolic blood pressure of the current user. Put the value of the systolic blood pressure of the current user into the formula for calculating the value of the diastolic blood pressure to calculate the value of the diastolic blood pressure of the current user directly, after the value of the systolic blood pressure of the current user is calculated.

A second object of the present invention is to provide a blood pressure measurement device. The blood pressure measurement device applying the above blood pressure measurement method to measure blood pressure, includes a microprocessor, a photoplethysmograph sensor, an electrocardiosignal sensor, a first analog-to-digital converter, a memory and a second analog-to-digital converter. The photoplethysmograph sensor is controlled by the microprocessor for sensing photoplethysmography signals of a subject. The electrocardiosignal sensor is controlled by the microprocessor for sensing electrocardiosignals of the subject. The first analog-to-digital converter is electrically connected between the photoplethysmograph sensor and the microprocessor. The photoplethysmography signals of the subject are converted into first digital signals by the first analog-to-digital converter. The memory is electrically connected with the first analog-to-digital converter by the microprocessor. The first digital signals are transmitted to and stored in the memory. The second analog-to-digital converter is electrically connected between the electrocardiosignal sensor and the microprocessor, and the electrocardiosignals of the subject are converted into second digital signals by the second analog-to-digital converter, and then the second digital signals are transmitted to and stored in the microprocessor.

A third object of the present invention is to provide a blood pressure measurement device which includes means for sampling and storing photoplethysmography signals and electrocardiosignals from plural different subjects; means for measuring blood pressure of plural different subjects to get a plurality of values of systolic blood pressure and diastolic blood pressure; means for synchronizing the electrocardiosignals and the photoplethysmography signals of each of the different subjects, and calculating a time interval between an electrocardiosignal feature point and a photoplethysmography signal feature point of each of the different subjects; means for establishing a formula for calculating the value of the systolic blood pressure of each of the subjects: SBP=a1×PWV+b1×BMI+c1, SBP being the value of the systolic blood pressure of each of the subjects; PWV being a pulse wave velocity, PWV=Height/(2×PTT), PTT being a pulse transit time, namely the time interval between the electrocardiosignal feature point and the photoplethysmography signal feature point of each of the subjects, Height being a height of each of the subjects; BMI being a body mass index of each of the subjects; a1 and b1 being two different coefficients, c1 being a constant; means for calculating a1, b1 and c1 by means of obtained values of the systolic blood pressure, PTT, Height and BMI of the different subjects applying a predicted model of monadic linear regression analysis method; means for establishing a formula for calculating the value of the diastolic blood pressure of each of the subjects: DBP=d1×SBP+e1, DBP being the value of the diastolic blood pressure of each of the subjects, SBP being the value of the systolic blood pressure of each of the subjects, d1 and e1 being respectively two different coefficients; means for calculating d1 and e1 by means of the obtained values of the systolic blood pressure and obtained values of the diastolic blood pressure of the different subjects applying the predicted model of monadic linear regression analysis method; means for sampling and storing photoplethysmography signals and electrocardiosignals of a current user, synchronizing the electrocardiosignals and the photoplethysmography signals of the current user, calculating a time interval between an electrocardiosignal feature point and a photoplethysmography signal feature point of the current user, values of Height and BMI of the current user, and putting the time interval between the electrocardiosignal feature point and the photoplethysmography signal feature point of a current user into the formula for calculating the value of the systolic blood pressure of the current user; and means for putting the value of the systolic blood pressure of the current user into the formula for calculating the value of the diastolic blood pressure to calculate the value of the diastolic blood pressure of the current user directly, after the value of the systolic blood pressure of the current user being calculated.

As described above, when the blood pressure measurement method is applied in the blood pressure measurement device to measure the blood pressure of the current user, the formula for calculating the value of the systolic blood pressure: SBP=a1×PWV+b1×BMI+c1 and the formula for calculating the value of the diastolic blood pressure: DBP=d1×SBP+e1 are established, a1, b1, c1, d1 and e1 are calculated by means of the obtained values of the systolic blood pressure, the diastolic blood pressure, PTT, Height and BMI of the different subjects applying the predicted model of monadic linear regression analysis method, the formulas: SBP=a1×PWV+b1×BMI+c1 and DBP=d1×SBP+e1 are written to the microprocessor. The photoplethysmograph sensor samples the photoplethysmography signals of the current user, and the electrocardiosignal sensor samples the electrocardiosignals of the current user, calculate the time interval between the electrocardiosignal feature point and the photoplethysmography signal feature point of the current user, the values of Height and BMI of the current user, and the time interval between the electrocardiosignal feature point and the photoplethysmography signal feature point of the current user is put into the formula for calculating the value of the systolic blood pressure of the current user by the microprocessor, so the value of the systolic blood pressure of the current user is capable of being calculated, and the value of the systolic blood pressure of the current user is put into the formula for calculating the value of the diastolic blood pressure by the microprocessor to calculate the value of the diastolic blood pressure of the current user directly.

### BRIEF DESCRIPTION OF THE DRAWINGS

The present invention will be apparent to those skilled in the art by reading the following description, with reference to the attached drawings, in which:

FIG. 1 is a flow chart of a blood pressure measurement method applied in a blood pressure measurement device in accordance with an embodiment of the present invention;

FIG. 2 is a block diagram of the blood pressure measurement device of FIG. 1;

FIG. 3 is a data sheet of values of diastolic blood pressure and systolic pressure of different subjects measured by sphygmomanometers;

FIG. 4 is a diagram of values of d1 and e1 in a diastolic blood pressure numerical formula by the blood pressure measurement device applying a predicted model of monadic linear regression analysis method; and

FIG. 5 is a graph of values of PPT calculated by the blood pressure measurement device of FIG. 1 applying the blood pressure measurement method.

### DETAILED DESCRIPTION OF THE PREFERRED EMBODIMENT

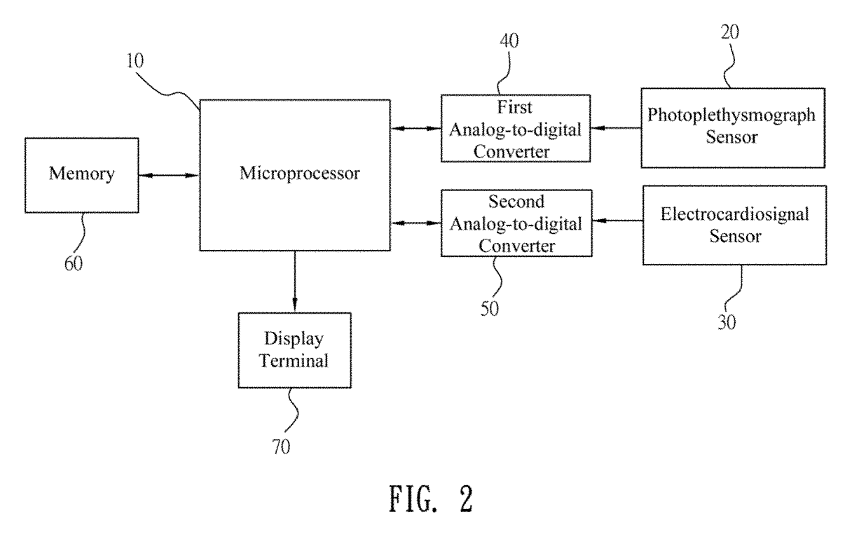

With reference to FIG. 2, a blood pressure measurement method and a blood pressure measurement device 100 in accordance with an embodiment of the present invention are shown. The blood pressure measurement method is applied in the blood pressure measurement device 100. The blood pressure measurement device 100 includes a microprocessor 10, a photoplethysmograph sensor 20, an electrocardiosignal sensor 30, a first analog-to-digital converter 40, a second analog-to-digital converter 50, a memory 60, a display terminal 70 and so on. The photoplethysmograph sensor 20, the electrocardiosignal sensor 30, the first analog-to-digital converter 40, the second analog-to-digital converter 50, the memory 60 and the display terminal 70 are electrically connected with the microprocessor 10.

The first analog-to-digital converter 40 is electrically connected between the photoplethysmograph sensor 20 and the microprocessor 10. The memory 60 is electrically connected with the first analog-to-digital converter 40 by the microprocessor 10. The photoplethysmograph sensor 20 is controlled by the microprocessor 10 for sensing photoplethysmography (PPG) signals of a subject. The photoplethysmography signals of the subject are converted into first digital signals by the first analog-to-digital converter 40, and then the first digital signals are transmitted to and stored in the memory 60.

Referring to FIG. 2, the second analog-to-digital converter 50 is electrically connected between the electrocardiosignal sensor 30 and the microprocessor 10. The electrocardiosignal sensor 30 is controlled by the microprocessor 10 for sensing electrocardiosignals of the subject, and the electrocardiosignals of the subject are converted into second digital signals by the second analog-to-digital converter 50, and then the second digital signals are transmitted to and stored in the memory 60.

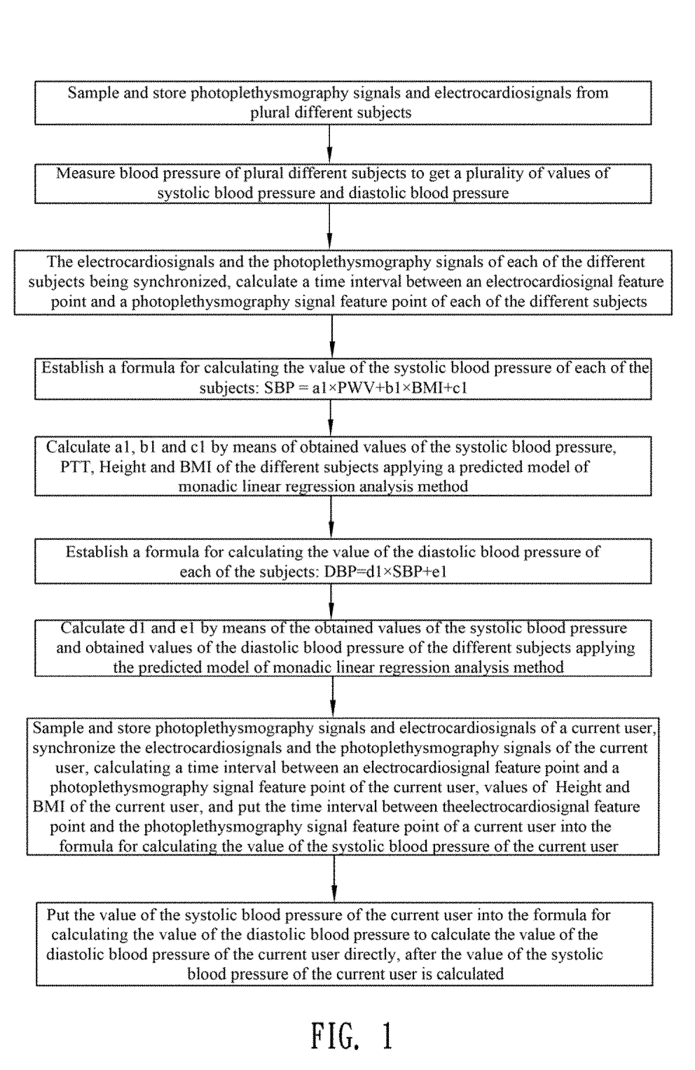

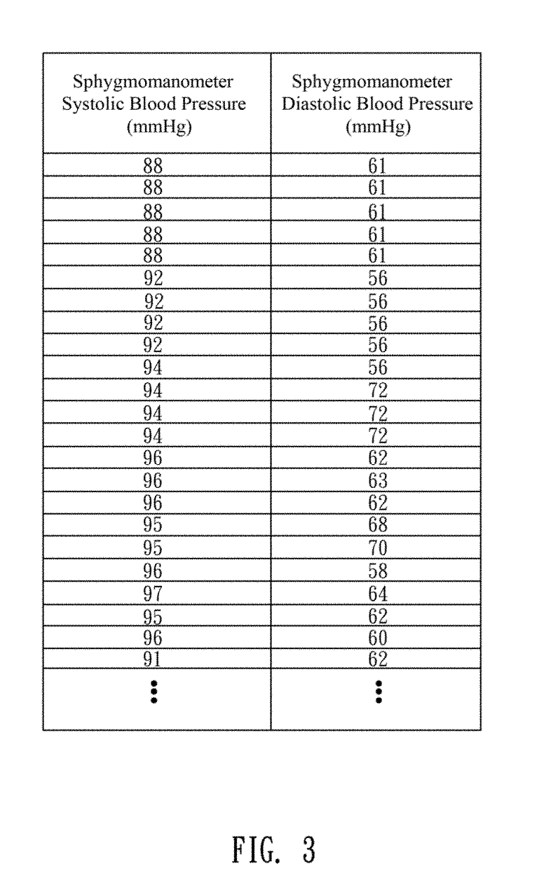

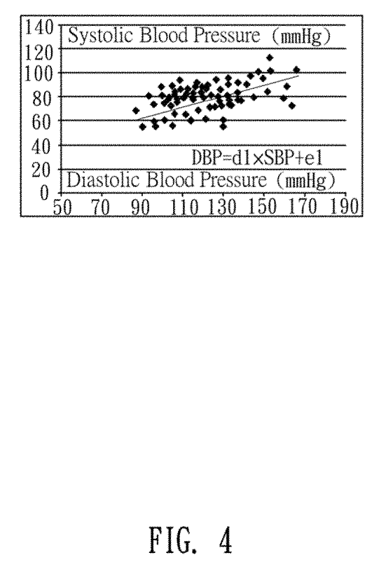

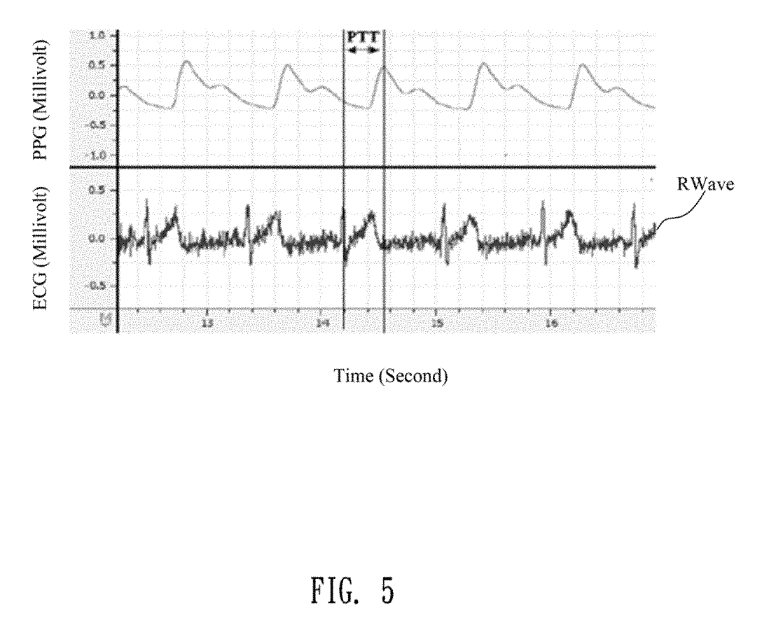

Referring to FIG. 1 to FIG. 5, specific steps of the blood pressure measurement method being applied in the blood pressure measurement device 100 to measure blood pressure of a current user are described as follows.

Firstly, the photoplethysmograph sensor 20 samples the photoplethysmography signals from plural different subjects, the first analog-to-digital converter 40 converts the photoplethysmography signals into the first digital signals, and then the first digital signals are stored in the memory 60. The electrocardiosignal sensor 30 samples the electrocardiosignals of the different subjects, the second analog-to-digital converter 50 converts the electrocardiosignals into the second digital signals, and then the second digital signals are stored in the memory 60. In this embodiment, a collecting position of the photoplethysmograph sensor 20 is a wrist of the subject.

Secondly, measure the blood pressure of the plural different subjects by sphygmomanometers to get a plurality of values of systolic blood pressure and diastolic blood pressure. The blood pressure of each of the subjects includes the systolic blood pressure and the diastolic blood pressure.

Thirdly, synchronize the electrocardiosignals and the photoplethysmography signals of each of the different subjects, and calculate a time interval between an electrocardiosignal feature point and a photoplethysmography signal feature point of each of the different subjects. In this embodiment, the time interval between the electrocardiosignal feature point and the photoplethysmography signal feature point of each of the subjects is a time interval between a peak value of an R wave of the electrocardiosignals of each of the subjects and a peak value of a photoplethysmography pulse wave of each of the subjects measured at the time of synchronizing the electrocardiosignals and the photoplethysmography signals of each of the subjects.

Fourthly, establish a formula for calculating the value of the systolic blood pressure (SBP) of each of the subjects by means of the value of the systolic blood pressure being linearly correlated with a pulse wave velocity and a body mass index of each of the subjects. The formula for calculating the value of the systolic blood pressure is shown as follows: SBP=a1×PWV+b1×BMI+c1. SBP is the value of the systolic blood pressure of each of the subjects; PWV is a pulse wave velocity, PWV=Height/(2×PTT), Height/2 is a distance between a heart and the wrist of each of the subjects, PTT is a pulse transit time, namely the time interval between the electrocardiosignal feature point and the photoplethysmography signal feature point of each of the subjects, Height is a height of each of the subjects; BMI is a body mass index of each of the subjects; a1 and b1 are two different coefficients, c1 is a constant.

Fifthly, calculate a1, b1 and c1 by means of obtained values of the systolic blood pressure, PTT, Height and BMI of the different subjects applying a predicted model of monadic linear regression analysis method. After a1, b1 and c1 are calculated, the formula: SBP=a1×PWV+b1×BMI+c1 is written in the microprocessor 10, SBP is an unknown number; a1, b1, c1 are known numbers.

Sixthly, establish a formula for calculating the value of the diastolic blood pressure (DBP) of each of the subjects. The formula for calculating the value of the diastolic blood pressure is shown as follows: DBP=d1×SBP+e1. DBP is the value of the diastolic blood pressure of each of the subjects; SBP is the value of the systolic blood pressure of each of the subjects; d1 and e1 are respectively two different coefficients.

Seventhly, calculate d1 and e1 by means of the obtained values of the systolic blood pressure and obtained values of the diastolic blood pressure of the different subjects applying the predicted model of monadic linear regression analysis method. After d1 and e1 are calculated, the formula: DBP=d1×SBP+e1 is written in the microprocessor 10, SBP and DBP are unknown numbers; d1 and e1 are known numbers.

Eighthly, the photoplethysmograph sensor 20 samples photoplethysmography signals of the current user and stores the photoplethysmography signals of the current user in the memory 60. The electrocardiosignal sensor 30 samples electrocardiosignals of the current user and stores the electrocardiosignals of the current user. Synchronize the electrocardiosignals and the photoplethysmography signals of the current user. Calculate a time interval between an electrocardiosignal feature point and a photoplethysmography signal feature point of the current user, namely, a time interval between a peak value of an R wave of the electrocardiosignals of the current user and a peak value of a photoplethysmography pulse wave of the current user. Values of Height and BMI of the current user, and put the time interval between the electrocardiosignal feature point and the photoplethysmography signal feature point of the current user into the formula for calculating a value of a systolic blood pressure (SBP) of the current user by the microprocessor 10, so the value of the systolic blood pressure of the current user is capable of being calculated.

Ninthly, put the value of the systolic blood pressure of the current user into the formula for calculating the value of the diastolic blood pressure by the microprocessor 10 to calculate the value of the diastolic blood pressure of the current user directly, after the value of the systolic blood pressure of the current user is calculated.

The calculated values of the systolic blood pressure and the diastolic blood pressure got by the microprocessor 10 of the blood pressure measurement device 100 are transmitted to a display terminal 70 to be displayed, so the blood pressure of the current user is measured. In this embodiment, the display terminal 70 is capable of being the blood pressure measurement device 100 with a displayer, a cell phone or other intelligent device. The display terminal 70 is not limited to be a visual display device, and the display terminal 70 is also able to be an audio display device and so on.

As described above, when the blood pressure measurement method is applied in the blood pressure measurement device 100 to measure the blood pressure of the current user, the formula for calculating the value of the systolic blood pressure: SBP=a1×PWV+b1×BMI+c1 and the formula for calculating the value of the diastolic blood pressure: DBP=d1×SBP+e1 are established, a1, b1, c1, d1 and e1 are calculated by means of the obtained values of the systolic blood pressure, the diastolic blood pressure, PTT, Height and BMI of the different subjects applying the predicted model of monadic linear regression analysis method, the formulas: SBP=a1×PWV+b1×BMI+c1 and DBP=d1×SBP+e1 are written to the microprocessor 10. The photoplethysmograph sensor samples the photoplethysmography signals of the current user, and the electrocardiosignal sensor 30 samples the electrocardiosignals of the current user, calculate the time interval between the electrocardiosignal feature point and the photoplethysmography signal feature point of the current user, the values of Height and BMI of the current user, and the time interval between the electrocardiosignal feature point and the photoplethysmography signal feature point of the current user is put into the formula for calculating the value of the systolic blood pressure of the current user by the microprocessor 10, so the value of the systolic blood pressure of the current user is capable of being calculated, and the value of the systolic blood pressure of the current user is put into the formula for calculating the value of the diastolic blood pressure by the microprocessor 10 to calculate the value of the diastolic blood pressure of the current user directly.

What is claimed is:

1. A blood pressure measurement method, comprising the steps of:
sampling and storing photoplethysmography signals and electrocardiosignals from plural different subjects;
measuring blood pressure of plural different subjects to get a plurality of values of systolic blood pressure and diastolic blood pressure;
synchronizing the electrocardiosignals and the photoplethysmography signals of each of the different subjects, and calculating a time interval between an electrocardiosignal feature point and a photoplethysmography signal feature point of each of the different subjects;
establishing a formula for calculating the value of the systolic blood pressure of each of the subjects: SBP=a1×PWV+b1×BMI+c1, SBP being the value of the systolic blood pressure of each of the subjects; PWV being a pulse wave velocity, PWV=Height/(2×PTT), PTT being a pulse transit time, namely the time interval between the electrocardiosignal feature point and the photoplethysmography signal feature point of each of the subjects, Height being a height of each of the subjects; BMI being a body mass index of each of the subjects; a1 and b1 being two different coefficients, c1 being a constant;
calculating a1, b1 and c1 by means of obtained values of the systolic blood pressure, PTT, Height and BMI of the different subjects applying a predicted model of monadic linear regression analysis method;
establishing a formula for calculating the value of the diastolic blood pressure of each of the subjects: DBP=d1×SBP+e1, DBP being the value of the diastolic blood pressure of each of the subjects, SBP being the value of the systolic blood pressure of each of the subjects, d1 and e1 being respectively two different coefficients;
calculating d1 and e1 by means of the obtained values of the systolic blood pressure and obtained values of the diastolic blood pressure of the different subjects applying the predicted model of monadic linear regression analysis method;
sampling and storing photoplethysmography signals and electrocardiosignals of a current user, synchronizing the electrocardiosignals and the photoplethysmography signals of the current user, calculating a time interval between an electrocardiosignal feature point and a photoplethysmography signal feature point of the current user, values of Height and BMI of the current user, and putting the time interval between the electrocardiosignal feature point and the photoplethysmography signal feature point of a current user into the formula for calculating the value of the systolic blood pressure of the current user; and
putting the value of the systolic blood pressure of the current user into the formula for calculating the value of the diastolic blood pressure to calculate the value of the diastolic blood pressure of the current user directly, after the value of the systolic blood pressure of the current user being calculated.

2. The blood pressure measurement method as claimed in claim 1, wherein the time interval between the electrocardiosignal feature point and the photoplethysmography signal feature point of each of the subjects is a time interval between a peak value of an R wave of the electrocardiosignals of each of the subjects and a peak value of a photoplethysmography pulse wave of each of the subjects measured at the time of synchronizing the electrocardiosignals and the photoplethysmography signals of each of the subjects.

3. The blood pressure measurement method as claimed in claim 1, wherein after a1, b1 and c1 are calculated, the formula: SBP=a1×PWV+b1×BMI+c1 is written in a microprocessor, SBP is an unknown number; a1, b1, c1 are known numbers.

4. The blood pressure measurement method as claimed in claim 1, wherein after d1 and e1 are calculated, the formula: DBP=d1×SBP+e1 is written in a microprocessor, SBP and DBP are unknown numbers; d1 and e1 are known numbers.

5. A blood pressure measurement device applying a blood pressure measurement method to measure blood pressure, comprising:
a microprocessor;
a photoplethysmograph sensor controlled by the microprocessor for sensing photoplethysmography signals of a subject;
an electrocardiosignal sensor controlled by the microprocessor for sensing electrocardiosignals of the subject;
a first analog-to-digital converter electrically connected between the photoplethysmograph sensor and the microprocessor, the photoplethysmography signals of the subject being converted into first digital signals by the first analog-to-digital converter;
a memory electrically connected with the first analog-to-digital converter by the microprocessor, the first digital signals being transmitted to and stored in the memory; and
a second analog-to-digital converter electrically connected between the electrocardiosignal sensor and the microprocessor, the electrocardiosignals of the subject being converted into second digital signals by the second analog-to-digital converter, and then the second digital signals being transmitted to and stored in the microprocessor.

6. The blood pressure measurement device as claimed in claim 1, further comprising a display terminal, calculated values of systolic blood pressure and diastolic blood pressure got by the microprocessor are transmitted to the display terminal to be displayed.

7. A blood pressure measurement device, comprising:
means for sampling and storing photoplethysmography signals and electrocardiosignals from plural different subjects;
means for measuring blood pressure of plural different subjects to get a plurality of values of systolic blood pressure and diastolic blood pressure;
means for synchronizing the electrocardiosignals and the photoplethysmography signals of each of the different subjects, and calculating a time interval between an electrocardiosignal feature point and a photoplethysmography signal feature point of each of the different subjects;
means for establishing a formula for calculating the value of the systolic blood pressure of each of the subjects: SBP=a1×PWV+b1×BMI+c1, SBP being the value of the systolic blood pressure of each of the subjects; PWV being a pulse wave velocity, PWV=Height/(2×PTT), PTT being a pulse transit time, namely the time interval between the electrocardiosignal feature point and the photoplethysmography signal feature point of each of the subjects, Height being a height of each of the subjects; BMI being a body mass index of each of the subjects; a1 and b1 being two different coefficients, c1 being a constant;
means for calculating a1, b1 and c1 by means of obtained values of the systolic blood pressure, PTT, Height and BMI of the different subjects applying a predicted model of monadic linear regression analysis method;
means for establishing a formula for calculating the value of the diastolic blood pressure of each of the subjects: DBP=d1×SBP+e1, DBP being the value of the diastolic blood pressure of each of the subjects, SBP being the value of the systolic blood pressure of each of the subjects, d1 and e1 being respectively two different coefficients;
means for calculating d1 and e1 by means of the obtained values of the systolic blood pressure and obtained values of the diastolic blood pressure of the different subjects applying the predicted model of monadic linear regression analysis method;
means for sampling and storing photoplethysmography signals and electrocardiosignals of a current user, synchronizing the electrocardiosignals and the photoplethysmography signals of the current user, calculating a time interval between an electrocardiosignal feature point and a photoplethysmography signal feature point of the current user, values of Height and BMI of the current user, and putting the time interval between the electrocardiosignal feature point and the photoplethysmography signal feature point of a current user into the formula for calculating the value of the systolic blood pressure of the current user; and
means for putting the value of the systolic blood pressure of the current user into the formula for calculating the value of the diastolic blood pressure to calculate the value of the diastolic blood pressure of the current user directly, after the value of the systolic blood pressure of the current user being calculated.
